(19) United States
(12) Patent Application Publication
(10) Pub. No.: US 2007/0088266 A1
(43) Pub. Date: Apr. 19, 2007

(54) Liquid transfer apparatus and control method therefor

(75) Inventors: Kazuo Yamauchi, Nishinomiya-shi (JP); Takayoshi Sawano, Nishinomiya-shi (JP); Noriaki Yamamoto, Nishinomiya-shi (JP)

Correspondence Address: Alfred A. Stadnicki;Antonelli, Terry, Stout & Kraus, LLP, 1300 North Seventeenth St., Suite 1800, Arlington, VA, 22209, US

(73) Assignee: FURUNO ELECTRIC COMPANY, LIMITED, NISHINOMIYA-CITY (JP)

(21) Appl. No.: 11/496,638

(22) Filed: Aug. 1, 2006

(30) Foreign Application Priority Data
Aug. 3, 2005 (JP) 2005-224903

(51) Int. Cl. A61M 37/00

(52) U.S. Cl. 604/131

(57) ABSTRACT

A residual liquid quantity in a syringe is reduced to the minimum. There are detected with high precision malfunctions due to chokes in pipes and incomplete switchings of an electromagnetic valve and a broken valve without a contamination problem produced. When a syringe pump is powered on, a motor is driven at low speed so that liquid in a syringe is discharged completely. When a desynchronization of the motor is detected by comparing motor driving pulses obtained by a rotary encoder with motor driving pulses supplied to the motor, a stop signal is supplied to a motor driver and the position of a rod is memorized as the origin of the syringe pump in an origin sensor. While the syringe pump is in a normal operating state, the position of the rod is detected to be at the origin to supply a stop signal to the motor driver.

### BACKGROUND OF THE INVENTION

The present invention relates to a liquid transfer apparatus or to a control method for the liquid transfer apparatus which draws liquid into a syringe and discharges the liquid from the syringe by respectively pulling and pushing a syringe tip in the syringe.

Conventionally, there has been used a liquid transfer apparatus such as a syringe when a quantity of liquid is drawn from a bottle into a tubing to be transferred to another tubing through an electromagnetic valve changing liquid flow paths (refer to Japanese patent 3475032 and Japanese Laid-Open Patent No. 2001-224681). With a liquid transfer apparatus such as a syringe pump, a mechanical origin sensor has been generally used to determine the origin point of the syringe pump (refer to Japanese Laid-Open Patent No. 8-220106).

Further, in transferring liquid, when there are caused malfunctions due to chokes in pipes or a broken or defective electromagnetic valve, there will come up in operating the syringe pump problems as liquid leakages or pipe explosions. A pressure sensor has been installed in the pipes to monitor pressures therein so that there have been prevented problems from being caused as pipe chokes and due to a broken or defective electromagnetic valve (refer to Japanese Laid-Open Patent No. 8-220106).

Now, a syringe pump is shown in FIG. 3 as an example of a liquid transport apparatus having been conventionally used. FIG. 3 is a schematic illustration of a prior-art syringe pump. As shown in FIG. 3, a syringe pump 1 is comprised with a syringe 2, a syringe tip 3, driving mechanism 10 and a pipe arrangement 20. The syringe 2 is fixed by a fixing mechanism that is not illustrated. The syringe tip 3 is contained in the syringe 2 and is pulled downward or pushed upward by the driving mechanism 10 to draw the liquid 4 into or discharge the liquid 4 from the syringe 2. With this syringe pump 1, the syringe tip 3 is pulled downward by the driving mechanism 10 as will be explained hereinafter so that the liquid 4 in the bottle 5 is drawn into the syringe 2 through the tubing 21a from the bottle 5. Then an electromagnetic valve 23 is switched and the syringe tip 3 is pushed upward so that the liquid 4 is transported to the other tubing 21b. The driving mechanism 10 is comprised with a rod 11 connected to the syringe tip 3, a motor 12 for driving the rod 11 and a motor driver 13 for controlling the motor 12. The rod 11 is provided in parallel with the syringe 2, is fixed to a belt 15 which has been extended and wound over two pulleys 14 and is moved by the motor 12. The belt 15 is driven by the motor 12 to move in parallel with the syringe 2. A stepping motor (a pulse motor) is used as the motor 12. The motor driver 13 is connected with an origin sensor 16. The origin sensor 16 stores as the origin point a spatial point determined when the syringe tip 3 is moved up to completely push out the liquid out of the syringe 2. The origin sensor 16 transmits a stop signal when it detects the origin point. It is to be noted that the origin of origin sensor 16 is adjusted manually. The motor driver 13 transmits drive signals to the motor 12 in a normal operating mode to control the motor 12.

The pipe arrangement 20 is comprised with a tubing 21 connected with the uppermost portion of the syringe 2 by means of a tube connector 22, the electromagnetic valve 23 switching pipe connections to cause a flow of the liquid 4 from the bottle 5 through the tubing 21a and to cause the other flow of the liquid through the tubing 21b to a desired part, and a pressure sensor 24 inserted in the tubing 21. The pressure sensor 24 monitors pressures in the tubing 21. When the sensor 24 detects a pressure above a predetermined value, the sensor 24 transmits a stop signal to the motor driver 13 in the drive mechanism 10, as it indicates a possibility that there has been caused malfunctions due to chokes in pipes or an inoperable, broken or defective electromagnetic valve.

However, with liquid transfer apparatuses, liquids for use as a chemical reagent are highly expensive. Thus, a consumed quantity of the liquid in use is desired to be reduced. With bottles, electromagnetic valves and pipes having been used in conventional liquid transfer apparatuses, factors such as arrangements and diameters are appropriately determined so that consumed amounts of a transported liquid are reduced to the minimum.

However, with adjustments of the origin sensor for conventional liquid transfer apparatuses, the origin point is adjusted manually to be fixed. Thus, preciseness of the position adjustment depends on individuals and needs mastery of skill. There have been such problems that the origin point was not detected if the position adjustment had been made to the utmost delicacy and changes such as change with time could not be coped with so that a residual liquid quantity (dead volume) in a syringe was not reduced to the minimum. In addition, with a prior art liquid transfer apparatus, a pressure sensor is inserted in a piping arrangement, which causes liquid to enter a pipe connected to the pressure sensor so that the pipe was not washed and thus a contamination was produced.

It is to be noted that there is another method to detect malfunctions such as chokes in pipes and due to defective switching operations of an electromagnetic valve or a broken valve by detecting pressure variations in the pipes which can be detected by checking measured value changes of the pressure sensor and also of the diameter of the pipes. With this method, there are such problems that highly precise detection is required, and thus reliability is inferior.

### SUMMARY OF THE INVENTION

Accordingly, the present invention provides a liquid transfer apparatus and a control method for the liquid transfer apparatus which is capable of reducing a residual liquid quantity in a syringe to the minimum, and detecting with high precision malfunctions due to chokes in pipes and incomplete or defective switchings of an electromagnetic valve or a broken electromagnetic valve without a contamination problem produced.

One feature of the present invention is to provide a liquid transfer apparatus which comprises a syringe tip in the syringe moved in two mutually opposite directions to draw liquid into the syringe and discharge the liquid therefrom, a motor for moving the syringe tip, a motor controller for supplying a signal to the motor to drive the motor and to stop the motor based on a stop signal supplied thereto, a motor movement signal generator for generating a signal representative of rotational movement of the shaft of the motor, and a desynchronization detector for detecting a desynchronization of the motor by comparing the signal representative of rotational movement of the shaft of the motor from the motor movement signal generator with the signal to drive the motor from the motor controller.

Another feature of the present invention is to provide a liquid transfer apparatus which further comprises a memory for storing a positional signal corresponding to a motor desynchronization occurred and a coincidence circuit for producing a stop signal when the positional signal from the memory coincides with a signal to drive the motor from.

Another feature of the present invention is to provide a liquid transfer apparatus which comprises a rod connected to a syringe tip in a syringe moved in two mutually opposite directions to draw liquid into a syringe and discharge the liquid from the syringe, a motor for moving the rod, a motor drive controller for supplying the motor with motor drive pulses to drive the motor and to stop the motor based on a stop signal supplied thereto, motor drive pulse measuring unit connected to the motor for measuring motor drive pulses to drive the motor, a desynchronization detector for detecting desynchronization of the motor by comparing the motor drive pulses measured and obtained by the motor drive pulse measuring unit with motor drive pulses supplied by the motor drive controller.

Further feature of the present invention is to provide a control method for a liquid transfer apparatus comprises supplying motor drive pulses to a motor for moving in two mutually opposite directions a rod connected to a syringe tip in a syringe to draw liquid into a syringe and discharge liquid from the syringe, measuring motor drive pulses for the motor, detecting a desynchronization of the motor by comparing the motor drive pulses with motor drive pulses supplied thereto, and stopping the motor being driven based on a stop signal supplied.

When the origin of a liquid transfer apparatus is adjusted, a motor is driven at a low speed to push the liquid out from a syringe completely, moving resistance between a syringe tip and the syringe will be increased considerably so that a motor desynchronization will be caused. Further, when a liquid transfer apparatus is in a normal operation state, malfunctions such as chokes in pipes or due to defective switching operations of an electromagnetic valve or a broken valve produce an excessive load to the liquid transfer apparatus so that a motor desynchronization occurs. Thus, when the origin of a liquid transfer apparatus is adjusted, there is compared motor drive pulses measured and obtained with motor drive pulses supplied thereto to detect a desynchronization of the motor. When the motor desynchronization is detected, there is automatically adjusted the origin of the liquid transfer apparatus for supplying a stop signal to stop the operating motor so that a residual liquid quantity in the syringe will be reduced to the minimum.

When a liquid transfer apparatus is in a normal operating state, a motor desynchronization is detected and thus a stop signal is supplied to stop the active motor, which results in detection of malfunctions such as chokes in pipes and due to incomplete switching operations of an electromagnetic valve or a defective valve. Further, it will not be necessary to have pressure sensors in the piping system of a liquid transfer apparatus, which will not produce a contamination problem. The liquid transfer apparatus according to the present invention further comprises an origin detector for supplying a stop signal to the motor drive controller when the origin detector detects a position of the rod to be at the origin of the liquid transfer apparatus having been memorized, wherein the motor drive controller drives the motor to move the rod so that the liquid in the syringe is discharged, and the origin detector memorizes the position of the rod as the origin of the liquid transfer apparatus when the desynchronization detector detects a desynchronization of the motor in adjusting the origin of the liquid transfer apparatus.

When the origin of a liquid transfer apparatus is adjusted, a motor is driven to discharge the liquid in a syringe completely, moving resistance between a syringe tip and the syringe wall will be increased considerably so that a motor desynchronization will be caused. Thus, in adjusting the origin of a liquid transfer apparatus, when the motor desynchronization means detects a desynchronization of the motor, i.e., when the origin detection means having memorized the position of the rod as the origin at a time instant when the liquid transfer apparatus discharges the liquid completely judges that a position of the rod coincides with the memorized origin, supplies a stop signal to the motor drive control means to stop the motor. Thus, the origin of the liquid transfer apparatus is automatically adjusted to reduce the liquid in the syringe to the minimum. Further, the origin of the liquid transfer apparatus is automatically adjusted, and it will not be necessary to adjust manually the origin, which has required mastery of skills. Changes with time can be coped with by further adjusting the origin of a liquid transfer apparatus frequently (for example, every time after each power-on). Thus, a residual liquid quantity in the syringe can be reduced to the minimum. Further, with a liquid transfer apparatus according to the present invention, it is also possible that the motor desynchronization detection means detects a motor desynchronization to provide the motor drive control means with a stop signal. Further, when a liquid transfer apparatus is in a normal operation state, malfunctions due to chokes in pipes or defective switching operations of an electromagnetic valve or a broken electromagnetic valve produce an excessive load to the liquid transfer apparatus so that a motor desynchronization occurs. When a liquid transfer apparatus is in a normal operating state, a motor desynchronization is detected and thus a stop signal is supplied to the motor drive control means to stop the active motor, which prevents malfunctions due to chokes in pipes and incomplete switching operations of an electromagnetic valve or a broken valve from being made. Further, it will not be necessary to have pressure sensors in the pipe arrangement of a liquid transfer apparatus, which will not produce a contamination problem. Further, in adjusting the origin of a liquid transfer apparatus, it is also possible that the motor desynchronization detection means detects a motor desynchronization to supply a stop signal to the motor drive control means so that the active motor is stopped when the liquid transfer apparatus discharges the liquid completely.

### BRIEF DESCRIPTION OF THE DRAWINGS

FIG. 1 is a schematic illustration showing a syringe pump according to a first embodiment of the invention.

FIG. 2 is a schematic illustration showing a syringe pump according to a second embodiment of the invention.

FIG. 3 is a schematic illustration showing a prior art syringe pump.

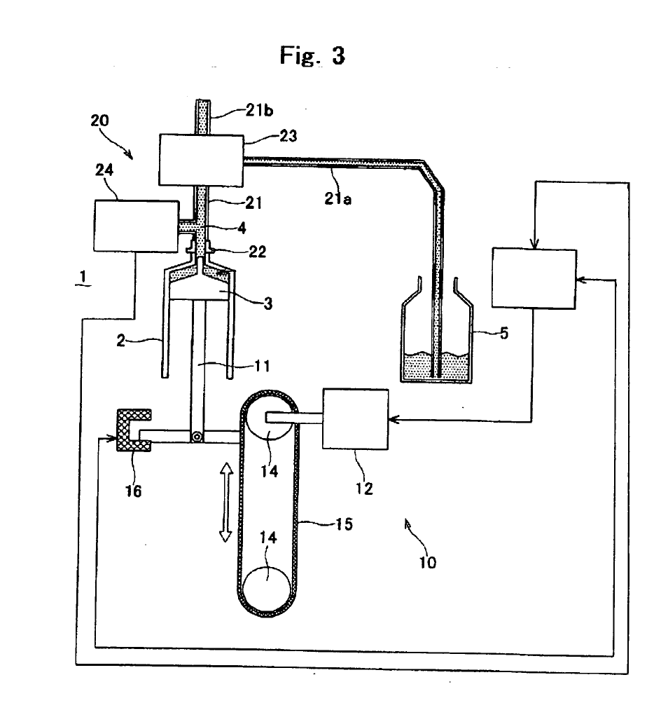

### PREFERRED EMBODIMENTS OF THE INVENTION

Referring to drawings, there will be explained hereinafter embodiments of a liquid transfer apparatus and control method for the liquid transfer apparatus according to the present invention.

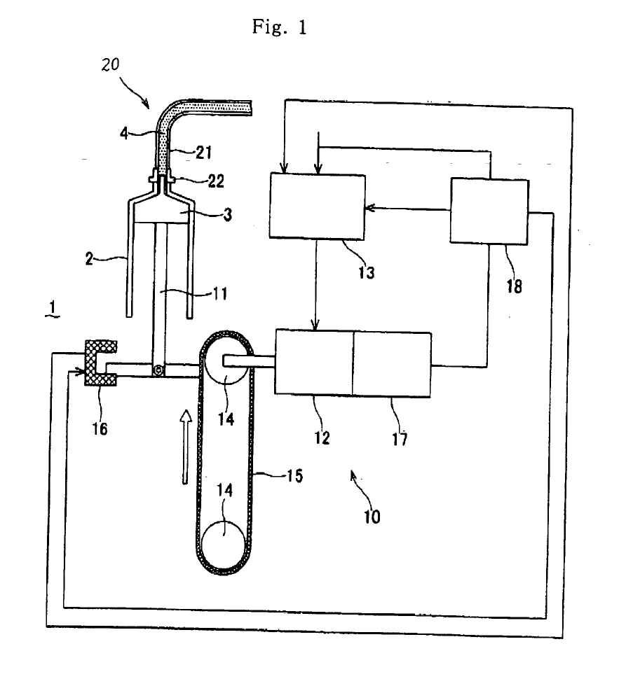

First, referring to FIG. 1, there will be explained a liquid transfer apparatus and a control method for the liquid transfer apparatus as a first embodiment according to the present invention. It is to be noted that a syringe pump is used as an example of a liquid transfer apparatus. A syringe pump 1 is comprised with a syringe 2 and a syringe tip 3, a driving mechanism 10, and a pipe arrangement 20. The syringe 2 is fixed by the fixing mechanism that is not illustrated. The syringe tip 3 is contained in the syringe 2, and is pulled or pushed by the moving mechanism 10 so that liquid 4 is drawn into the syringe 2 or is discharged therefrom. With this syringe pump 1, the syringe tip 3 is pulled by the mechanism 10 and plural tubes 21 (not shown) in the pipe arrangement 20 are switched to select a tubing by the electromagnetic valve (not shown) so that the liquid 4 is drawn into the syringe 2 or transported to another tubing.

The driving mechanism 10 is comprised with a rod 11 connected to a syringe tip 3, a motor 12 to move the rod 11, a motor driver (a motor drive control means) 13, a rotary encoder (motor drive pulse measurement means) 17 connected to the motor 12, a motor desynchronization detection circuit (desynchronization detection means) 18 for detecting a desynchronization of the motor 12, and an origin sensor (origin detection means) 16.

The rod 11 is arranged to be in parallel with the syringe 2 and is fixed to a belt 15 which is extended and wound over two rotatable pulleys 14. The pulley 14 is driven by the motor 12. The belt 15 is moved by the motor 12 in parallel with the vertical axis of the syringe 2. The syringe tip 3 at one end of the rod 11 is moved down or up in the syringe 2. A stepping motor (pulse motor) is used as the motor 12.

The desynchronization detection circuit 18 receives a signal for driving the motor 12 to rotate the shaft of the motor 12 and a signal representative of a rotated angle of the shaft of the motor 12 supplied from the rotary encoder 17. The desynchronization detection circuit 18 detects a motor desynchronization when the signal for driving the motor 12 does not coincide with the signal supplied from the rotary encoder 17. A rotary encoder 17 is connected to the motor 12, and produces a signal representative of an angle of the shaft of the motor 12. For instance, the rotary encoder 17 obtains motor drive pulses determined based on a rotation angle of the shaft of the motor 12. The drive pulse measurement means is not limited to the rotary encoder 17. There can be used other means which is capable of measuring and obtaining motor rotation—related pulses. The desynchronization detection circuit 18 receives motor drive pulses measured and obtained by the rotary encoder 17 and motor drive pulses for driving the motor 12, and detects a motor desynchronization when the respective two drive pulses from the two sources are different from each other.

The motor driver 13 controls operations of the motor 12, and provides the motor 12 with motor drive pulses to rotate the shaft of the motor 12 and also stops the motor 12 based on a stop signal supplied thereto. The motor driver 13 drives the motor 12 to discharge the liquid 4 in the syringe 2 completely. The syringe tip 3 is pushed against the syringe 2 so that move resistance therebetween will be increased considerably and thus a motor desynchronization will be caused. There is connected to the motor driver 13 the origin sensor 16 and the motor desynchronization detection circuit 18.

The origin sensor 16 supplies the motor driver 13 with a stop signal, when the sensor 16 detects that the rod 11 is at the origin point having been memorized beforehand. The origin sensor 16 is connected to the desynchronization detection circuit 18, and memorizes the position of the rod 11 as the origin of the syringe pump 1 when the detection circuit 18 detects in an origin adjustment process a desynchronization of the motor 12, i.e., when the syringe pump discharges the liquid 4 completely. It is desirable to conduct an origin point adjustment whenever the syringe pump 1 is switched on. The motor desynchronization detection circuit 18 supplies a stop signal to the motor driver 13 when the circuit 18 detects a desynchronization of the motor 12 in adjusting the origin, i.e., when the syringe pump 1 discharges the liquid 4 completely. It is to be noted that the motor desynchronization circuit 18 needs not to be connected to the motor driver 13. In this case, the desynchronization circuit 18 does not supply a stop signal to the motor driver 13 when the circuit 18 detects, in adjusting the origin point, a motor desynchronization, i.e., when the syringe pump 1 discharges the liquid completely.

The pipe arrangement 20 is comprised with a tubing 21 connected to the upper portion of the syringe 2 through the tube connector 22 and an electromagnetic valve (not shown) switching to select an appropriate one of the plumbing tubes (not shown).

Next, by referring to FIG. 1, there will be explained a control method of the syringe pump illustrated in the first embodiment of the present invention.

When it is determined that the origin is being adjusted, i.e., a power source is turned on, the motor driver 13 supplies drive signals to drive the motor 12 to be rotated at a low speed so that the syringe tip 3 is moved upward to discharge the liquid 4 completely (motor drive step). When the syringe tip 3 is pushed against the syringe 2 to discharge the liquid 4 completely, move resistance between the syringe 2 and the syringe tip 3 becomes higher so that a desynchronization of the motor 12 will be caused to halt the rotary encoder 17. And motor drive pulses are measured and obtained by the rotary encoder 17 (motor drive pulse measurement step). The motor drive pulses obtained by the rotary encoder 17 are compared with motor drive pulses supplied to the motor 12. If a measurement value of the rotary encoder 17 is different from a value of supplied motor drive pulse, a motor desynchronization with the motor 12 is detected by the desynchronization detection circuit 18 (motor desynchronization detection step).

When a motor desynchronization with the motor 12 is detected, a stop signal is transmitted to the motor driver 13 from the motor desynchronization circuit 18 to stop the rotation of the motor 12 and the position of the rod 11 of the syringe pump 1 is also memorized in the origin sensor 16. While the apparatus being in a normal operating condition, when the position of the rod 11 is detected to be at the origin by the origin sensor 16, a stop signal is supplied to the motor driver 13 from the origin sensor 16 to stop the rotation of the motor 12. (motor drive stop step). With the control method for a syringe pump according to the first embodiment of the invention as above—explained, there is memorized the position of the rod 11 as the origin of the syringe pump 1 by the origin sensor 16 when the syringe tip 3 discharges the liquid 4 in the syringe 2 completely. As a result, a liquid residual quantity (dead volume) in the syringe 2 will be minimized (refer to FIG. 1).

When there are used the syringe pump 1 according to the first embodiment of the invention and the syringe pump control method for the syringe pump 1 in the first embodiment, the motor 12 is driven to discharge the liquid 4 in the syringe 2 completely in adjusting the origin of the syringe pump so that a move and pushing resistance of the syringe tip 3 against the syringe 2 will be increased to result in a desynchronization of the motor 12. Thus, in adjusting the origin of the syringe pump 1, when the motor desynchronization detecting circuit 18 has detected a motor desynchronization of the motor 12, i.e., when the syringe pump 1 has discharged the liquid 4 completely, the origin sensor 16 has determined the corresponding position of the rod 11 as the origin of the syringe pump 1. When the origin sensor 16 determines the position of the rod 11 to be at the same position as the origin thereof, the sensor 16 supplies the motor driver 13 with a stop signal to stop the rotation of the motor 12 so that the origin of the syringe pump 1 is automatically adjusted and thus, a residual quantity of the liquid in the syringe 2 is reduced to the minimum. Further, the origin of the syringe pump 1 is automatically adjusted. Manual operations are not necessary to perform adjustments to determine the origin thereof requiring mastery of skills. Changes such as changes with time can be coped with by further adjusting the origin of the syringe pump 1 frequently (for example, one adjustment for each power-on operation). As a result, a liquid residual quantity in the syringe 2 can be reduced to the minimum.

Further, in adjusting the origin of the syringe pump 1, it is also possible that the motor desynchronization detection circuit 18 detects a motor desynchronization of the motor 12 to provide the drive control means with a stop signal so that the motor is stopped to be driven when the liquid transfer apparatus discharges the liquid completely.

With the embodiment of the invention, when there is detected by the origin sensor 16 a position of the rod or the syringe tip corresponding to a desynchronization of the motor (i.e., when the origin detection means, having memorized the position of the rod as the origin at a time instant when the liquid transfer apparatus discharges the liquid completely, judges that a position of the rod coincides with the memorized origin), the origin sensor 16 supplies a stop signal to the motor drive control means to stop the motor.

It is to be noted that the logic for performing the comparison or making the determination for the motor desynchronization or for producing the stop signal can be implemented using either hard wiring (e.g. a circuit) or programmed instructions (e.g. software executed on a processor).

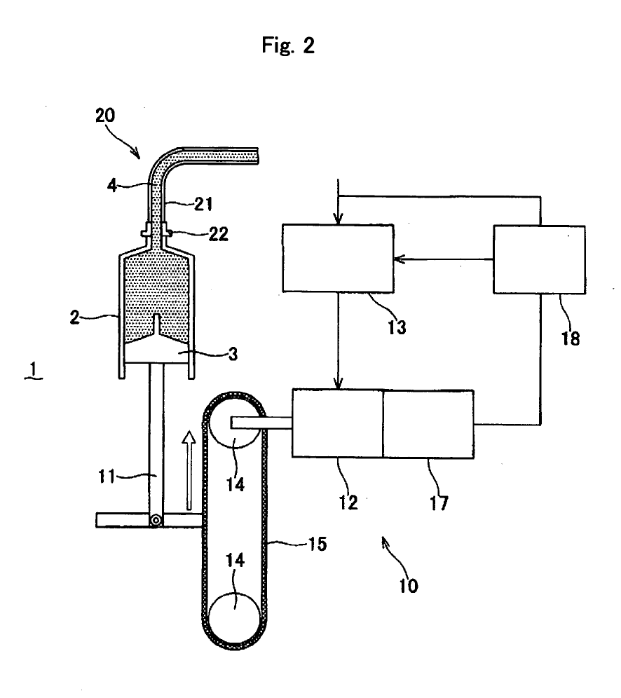

Referring to FIG. 2, there will be explained hereinafter a liquid transfer apparatus and a control method for the liquid transfer apparatus according to a second embodiment of the present invention.

FIG. 2 is a schematic illustration showing a syringe pump according to a second embodiment of the present invention. With this embodiment of the present invention, it is noted that a syringe pump is used as an example of a liquid transfer apparatus. As shown in FIG. 2, a syringe pump 1 is comprised with a syringe 2, a syringe tip 3 and driving mechanism 10, and a pipe system 20. The syringe 2 is fixed by a locking mechanism not illustrated. The syringe tip 3 is contained in the syringe 2, and is pulled downward or pushed upward by the driving mechanism 10 so that the liquid 4 is drawn into the syringe 2, or is discharged from the syringe 2. With this syringe pump 1, the syringe tip 3 is pulled by the moving mechanism 10 and plural plumbing tubes 21 (not shown) in the pipe arrangement 20 are switched to select one tubing thereof by the electromagnetic valve (not shown) so that liquid 4 is sucked or drawn into the syringe 2 or transported to a selected tubing.

The driving mechanism 10 is comprised with a rod 11, a motor 12 for driving the rod 11, a motor driver (motor drive control means) 13 for controlling drive and stop operations of the motor 12, a rotary encoder (motor drive pulse measurement means) 17 connected to the motor 12, and a motor desynchronization detection circuit (desynchronization detection means) 18 for detecting a desynchronization of the motor 12. The rod 11 is arranged to be in parallel with the syringe 2 and is fixed to a belt 15 which is extended and wound over two rotatable pulleys 14. The pulley 14 is rotated by the motor 12. The belt 15 is moved by the motor 12 in parallel with the syringe 2. A stepping motor (pulse motor) is used as the motor 12. A rotary encoder 17 is connected to the motor 12, and measures and obtains motor drive pulses determined by a rotational angle of the shaft of the motor 12. The drive pulse measurement means is not limited to the rotary encoder 17. There can be used other means which is capable of measuring and obtaining motor drive pulses. The motor desynchronization detection circuit 18 receives motor drive pulses measured and obtained by the rotary encoder 17 and motor drive pulses supplied from the motor driver 13, and detects a motor desynchronization when the two motor drive pulses from the two sources are different from each other. The motor driver 13 controls operation of the motor 12, and provides the motor 12 with motor drive pulses so that the motor 12 is driven and also stops the motor 12 based on a stop signal supplied thereto. A motor desynchronization detection circuit 18 is connected to the motor driver 13. The motor desynchronization detection circuit 18 supplies the motor driver 13 with a stop signal when it detects a motor desynchronization of the motor 12 in a normal operation mode.

The pipe arrangement 20 is constructed from a tubing 21 connected to the upper portion of the syringe 2 by means of a tube connector 22, and an electromagnetic valve not illustrated for switching a plurality of piping tubes (not shown) so that tube connections for liquid transfers are made between appropriate ones.

Next, referring to FIG. 2, there will be explained a control method for a syringe pump according to a second embodiment of the invention using the syringe pump in the above—explained second embodiment.

After the liquid 4 is entered into the syringe 2, the motor 12 is driven by drive signals from the motor driver 13 to move the syringe tip 3 upward so that the liquid 4 in the syringe 2 will be discharged. (motor drive step). When there are caused malfunctions due to chokes in pipes or abnormal switching operations of the electromagnetic valve or a broken electromagnetic valve, there will be produced an excessive load so that a signal representative of the rotation of the motor 12 will be different from a signal for driving the motor 12, i.e., the rotation of the motor is desynchronized with respect to drive pulses for driving the motor. Thus, a motor desynchronization occurs. Measured motor drive pulses are obtained by the rotary encoder 17 (motor drive pulse measurement step). Measured motor drive pulses obtained by the rotary encoder 17 are compared with motor drive pulses supplied to the motor 12. If the number of the pulses measured by the rotary encoder 17 is different from the corresponding one of the pulses supplied to the motor 12, a desynchronization of the motor 12 will be detected by the motor desynchronization detection circuit 18. When the motor desynchronization detection circuit 18 detects a motor desynchronization of the motor 12, a stop signal is supplied to the motor driver 13 from the motor desynchronization detection circuit 18 so that the motor 12 will be stopped (motor drive stop step). With the control method for controlling the syringe pump according to the second embodiment of the invention using the syringe pump in the above—explained second embodiment of the invention, malfunctions such as chokes in pipes or due to defective switching operations of an electromagnetic valve or a broken electromagnetic valve are detected with high precision by the motor desynchronization detection circuit 18 based on a motor desynchronization of the motor 12 so that liquid leakages from pipes or breakdowns thereof and the like will be prevented.

As explained above, with the syringe pump 1 according to the second embodiment of the invention or with the control method for the syringe pump used in the second embodiment, excessive loads are brought to the syringe pump 1 with malfunctions due to chokes in pipes 20 and the like so that the rotational operation of the motor 12 is desynchronized with respect to drive pulses supplied thereto. Thus, while the syringe pump 1 is normally driven, the motor desynchronization detection circuit 18 detects a motor desynchronization of the motor 12 to supply a stop signal to the motor driver 13 to stop the rotation of the motor 12 so that there will be prevented malfunctions due to chokes in the pipes 20 or defective switching operations of an electromagnetic valve or a broken electromagnetic valve. Further, it will not be necessary to have pressure sensors in the piping system 20 of syringe pump 1 installed so that there will be no contamination problems.

The present invention has been explained with preferred embodiments hereinbefore, but the present invention is not limited to the embodiments explained above. There are various other embodiments according to the present invention without departing from the spirit and scope of the invention. Further, with the present embodiment, operations by the arrangement of the invention and effects produced thereby have been described, but these operations and effects are examples, and the present invention is not limited to only the embodiments explained above. The arrangements of the embodiments of the present invention have been provided as examples. For instance, another embodiment of the invention is a combination of the first embodiment and the second embodiment of the invention explained above. There will be explained hereinafter a syringe pump 1 resulting from a combination of the first embodiment and the second embodiment of the invention.

The drive mechanism 10 of the syringe pump 1 resulting from a combination of the first embodiment and the second embodiment is similar to the one as shown in FIG. 1. The drive mechanism 10 is comprised with the rod 11 connected to the syringe tip 3, the motor 12 for driving the rod 11, the motor driver (motor control means) 13 for controlling a drive and a stop of the motor 12, a rotary encoder 17 connected to the motor 12 (motor drive pulse measuring means), the motor desynchronization detection circuit 18 for detecting a motor desynchronization of the motor 12 (motor desynchronization detection means), and the origin sensor 16 connected to the motor desynchronization detection circuit 18 (origin detection means). The origin sensor 16 detects the origin during a normal operation mode to supply a stop signal to the motor driver 13. The motor desynchronization detection circuit 18 detects a motor desynchronization of the motor 12 in adjusting the origin to supply the motor driver 13 with a stop signal, and makes the origin sensor 16 memorize the position of the rod 11 when a motor desynchronization of the motor 12 is detected, as the origin of the syringe pump 1. The motor desynchronization detection circuit 18 detects, during a normal operation mode, a motor desynchronization of the motor 12 to supply the motor driver 13 with a stop signal.

Next, there will be explained a control method for controlling a syringe pump 1 according to another embodiment resulting from a combination of the first embodiment and the second embodiment of the invention. In adjusting the origin of the syringe pump 1, i.e., at a time of turning the syringe pump 1 on, the motor 12 is moved at a low speed by means of the motor driver 13 to discharge the liquid 4 in the syringe 2 completely. A motor desynchronization of the motor 12 is detected by the motor desynchronization detection circuit 18 by comparing measured drive pulses obtained by the rotary encoder 17 with motor drive pulses supplied to the motor 12. When a motor desynchronization of the motor 12 is detected, a stop signal is supplied to the motor driver 13, and also the position of the rod 11 is stored automatically as the origin of the syringe pump 1 in the origin sensor 16. On the other hand, during a normal operational mode for the syringe pump 1, there are compared by the motor desynchronization detection circuit 18 measured motor drive pulses obtained by the rotary encoder 17 with motor drive pulses supplied to the motor 12 to detect a motor desynchronization of the motor 12 to supply a stop signal to the motor driver 13. Also, when the origin sensor 16 determines that the position of the rod 1 1 is at the origin, a stop signal is supplied to the motor driver 13. With the syringe pump resulting from a combination of the first embodiment and the second embodiment of the invention and the control method for controlling the combined syringe pump, there will be produced effects resulting from a syringe pump according to the first embodiment and from a control method for controlling the syringe pump with the first embodiment, and also effects resulting from a syringe pump according to the second embodiment and from a control method for controlling the syringe pump with the second embodiment.

What is claimed is:

1. A liquid transfer apparatus comprising:
a rod connected to a syringe tip in a syringe moved in two mutually opposite directions to draw liquid into the syringe and discharge the liquid therefrom;
a motor for moving the rod;
a motor drive controller for supplying motor drive pulses to the motor to drive the motor and to stop the motor based on a stop signal supplied thereto;
a motor drive pulse generating unit connected to the motor for obtaining motor drive pulses; and
a desynchronization detector for detecting a desynchronization of the motor by comparing the motor drive pulses obtained by the motor drive pulse generating unit with the motor drive pulses supplied by the motor drive controller.

2. The liquid transfer apparatus as claimed in claim 1 further comprising an origin detector for supplying a stop signal to the motor drive controller when the origin detector detects a position of the rod at the origin of the liquid transfer apparatus having been memorized, wherein the motor drive controller drives the motor to move the rod so that the liquid in the syringe is discharged, and the origin detector memorizes the position of the rod as the origin of the liquid transfer apparatus when the desynchronization detector detects a desynchronization of the motor in adjusting the origin of the liquid transfer apparatus.

3. The liquid transfer apparatus as claimed in claim 1 or claim 2 in which the desynchronization detector detects a desynchronization of the motor to supply a stop signal to the motor drive controller.

4. A control method for a liquid transfer apparatus comprising:
step for supplying motor drive pulses to a motor for moving in two mutually opposite directions a rod connected to a syringe tip in a syringe to draw liquid into the syringe and discharge the liquid therefrom;
step for obtaining motor pulses determined based on a rotational angle of the shaft of the motor;
step for detecting a desynchronization of the motor by comparing the motor drive pulses with motor pulses supplied thereto to produce a stop signal; and
step for stopping the motor being driven based on at least the stop signal.

5. A liquid transfer apparatus having a syringe comprising:
a syringe tip in the syringe moved in two mutually opposite directions to draw liquid into the syringe and discharge the liquid therefrom;
a motor for moving the syringe tip;
a motor controller for supplying a signal to the motor to drive the motor and to stop the motor based on a stop signal supplied thereto;
a motor movement signal generator for generating a signal representative of rotational movement of a shaft of the motor; and
a desynchronization detector for detecting a desynchronization of the motor by comparing the signal representative of rotational movement of the shaft of the motor from the motor movement signal generator with the signal to drive the motor from the motor controller.

6. The liquid transfer apparatus as claimed in claim 5 in which the desynchronization detector detects a desynchronization of the motor to supply a stop signal to the motor controller.

7. The liquid transfer apparatus as claimed in claim 5 further comprising a memory for storing a positional signal corresponding to a motor desynchronization occurred and a coincidence circuit for producing a stop signal when the positional signal from the memory coincides with a signal representative of a current position of the syringe tip.

8. A method of operating a syringe having a syringe tip movable to draw liquid into the syringe and discharge the liquid therefrom, comprising:
issuing a drive control signal corresponding to a desired amount of movement of the syringe tip;
moving the syringe tip responsive to the issued drive control signal;
generating a drive signal corresponding to an actual amount of movement of the syringe tip responsive to the issued drive control signal; and
comparing the generated drive signal with the issued drive control signal, to detect a desynchronization.

9. The method as claimed in claim 8, further comprising:
issuing a stop control signal to stop the movement of the syringe tip if the desyncronization is detected.

10. The method as claimed in claim 8, further comprising:
detecting, as the syringe tip is moved based on the issued drive control signal, a position corresponding to a current position of the syringe tip;
comparing the detected position with a predetermined position;
determining, based on the comparison, if the liquid in the syringe is completely discharged; and
issuing a stop control signal to stop the movement of the syringe tip if it is determined that the liquid in the syringe is completely discharged.
